(19) United States
(12) Patent Application Publication
(10) Pub. No.: US 2026/0130622 A1
(43) Pub. Date: May 14, 2026

(54) A System and Method of Early Diagnosis for Autism Spectrum Disorder

(71) Applicant: Natus Medical Incorporated, Middleton, WI (US)

(72) Inventors: Ching-Chang Kuo, Eugene, OR (US); Lalit Venkatesan, Celina, TX (US)

(73) Assignee: Natus Medical Incorporated, Middleton, WI (US)

(21) Appl. No.: 19/118,634

(22) PCT Filed: Oct. 4, 2023
(86) PCT No.: PCT/US2023/075969

§ 371 Date: Apr. 4, 2025

Related U.S. Application Data
(60) Provisional application No. 63/378,430, filed on Oct. 5, 2022.

(51) Int. Cl. A61B 5/00; A61B 5/38

(52) U.S. Cl. CPC A61B 5/4076; A61B 5/38; A61B 5/7267; A61B 5/7275; A61B 2503/045

(57) ABSTRACT

A method for early diagnosis of autism spectrum disorder (ASD) including determining an ASD risk index, determining an ASD brain index that includes administering at least one auditory brainstem response (ABR) test to an ear canal of a patient, producing a plurality of biomarkers and identifying at least one biomarker of the plurality of biomarkers as a potential ASD biomarker, and determining an ASD likelihood responsive to each of the ASD risk index and ASD brain index.

### FIELD OF THE INVENTION

This application is a national phase application of and claims priority under 35 U.S.C. § 371 of PCT U.S. Patent Application Ser. No. PCT/US23/75969 (Attorney Docket No. 4735.01187) filed on Oct. 4, 2023 and titled A System and Method of Early Diagnosis for Autism Spectrum Disorder, which in turn is a PCT application of and claims priority under 35 U.S.C. § 120 of U.S. Patent Application Ser. No. 63/378,430 (Attorney Docket No. 4735.01110) filed on Oct. 5, 2022 and titled A System and Method of Early Diagnosis for Autism Spectrum Disorder. The contents of these applications are incorporated herein by reference.

### BACKGROUND

ASD is a neurodevelopmental disorder associated with impairment in cognition, language, social skills and presence of repetitive behaviors and restricted interests, which has a higher ratio in boys than girls (4:1). In 2020, CDC reported that 1 in 54 children is diagnosed with ASD, a 10% prevalence, in the U.S. Based on the estimation in ASD healthcare services, the most common direct costs include Applied Behavioral Analysis (ABA) Interventions at $46,000-47,500 at per year, clinical or at-home ABA exercises at $15,000 per year, and speech therapy, occupational therapy, and physical therapy at $12,000 per year. The cost of ASD care may rise to $461 billion by 2025 in the absence of effective interventions throughout the lifetime of the patient.

Because an infant's brain develops over time, positive outcomes from early intervention can be achieved if accurate diagnosis is performed under the age of 2. Reduction in the time gap when the infants/children can be screened and diagnosed, and when they are actually diagnosed, is crucial. This gap plays a vital role in enhancing long-term outcomes regarding daily living skills, social behavior, adaptive behavior, communication, and cognition. However, the average age of diagnosis is after the age of four, and such late diagnoses misses the most effective time to provide treatment and therapy, involving pharmacological, medical, dietary and technological interventions. Currently, no definitive biomarkers or early diagnostic tools are available to diagnose ASD in newborns. Accordingly, there is a need in the art for an early screening tool for the diagnosis of ASD in newborns.

An integration of universal newborn hearing screening (UNHS), auditory brainstem response (ABR) test, and chirp stimuli is applied as an innovative diagnostic tool to offer all newborns and toddler the opportunity for accurate and early diagnosis of ASD. To reduce variation in interpretation between clinicians, accurate machine learning (ML) models may be developed to automate the process of analyzing ABR waveforms at suprathreshold levels. This screening approach is safe, efficient, and cost-effective, which offers promising techniques to determine ASD characteristics at earliest possible age, there by early intervention and appropriate therapy can benefit children with ASD and increase their chances to develop essential social and communication abilities during infancy.

One of the limitations is that the UNHS testing requires low intensities (1 intensity level in 35 dB), which makes it harder to differentiate most wave components. To address this issue, application of a chirp stimulus is an effective way to dramatically increase waveform amplitudes at the same intensity. A chirp is useful to detect early evoked responses with higher neural synchronization, larger wave V amplitudes, higher signal noise ratio (SNR), extensive frequency band (500-4 k Hz) coverage and shorter time test. With larger amplitude ABRs, it detects thresholds at low intensity level efficiently and more effectively in newborn and toddler screening and diagnosis.

Although the etiology of ASD is still unclear, articles suggest that there is a strong link between ASD and auditory deficits. Especially, children with ASD may suffer different degree of auditory dysfunction, such as central auditory processing disorders, atypical hemispheric lateralization, auditory oscillatory impairments in pre- & post-stimulus superior temporal gyrus (STG), and prolonged STG auditory responses. Children with ASD have difficulties in processing auditory information attribute to abnormal brain maturation and approximately 6% of children with hearing loss co-occur ASD. Additionally, hearing deficits in ASD may develop as hyperacusis, suggesting hypersensitivity to certain sounds or to noises. For instance, they respond to high-pitched sounds, understand music, and pay no attention to lower-pitched sounds or changes in speech. As a result, a wide spectrum of manifestations in ASD and hearing deficits contribute to the invention of this screening tool.

Furthermore, the brainstem is mainly developed in infancy and auditory brainstem response (ABR) is widely measure in newborn hearing screening. ABR measurement reflects a stimulus to detect auditory neuropathy using an electrical potential signal elicited from the scalp of the brain.

### SUMMARY OF THE INVENTION

A method and system for early diagnosis of ASD is presented herein. The method comprises identifying biomarkers indicative of ASD via hearing screening. A goal of the invention is to apply a screening tool in the early diagnosis of ASD. Particularly, ASD may be diagnosed by identifying the biomarkers of delayed brainstem response to auditory stimuli.

An embodiment of the invention is directed to an integration UNHS, auditory brainstem response (ABR) test, and chirp stimuli being applied as an innovative diagnostic tool to offer all newborns and toddler the opportunity for accurate and early diagnosis of ASD. To reduce variation in interpretation between clinicians, accurate Machine Learning (ML) models are developed to automate the process of analyzing ABR waveforms at suprathreshold levels. This screening approach is safe, efficient, and cost-effective, which offers promising techniques to determine ASD characteristics at earliest possible age, there by early intervention and appropriate therapy can benefit children with ASD and increase their chances to develop essential social and communication abilities during infancy.

### BRIEF DESCRIPTION OF THE DRAWINGS

FIG. 1 is a schematic view of a screening tool according to an embodiment of the invention.

FIG. 2 is a graph depicting an ABR response of a newborn according to an embodiment of the invention.

FIG. 3 is a graph depicting ABR responses for chirp and click ABR tests according to embodiments of the invention.

FIG. 4 is a flowchart illustrating a method of ASD diagnosis according to an embodiment of the invention.

FIG. 5 is a schematic view of a system for diagnosing ASD according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

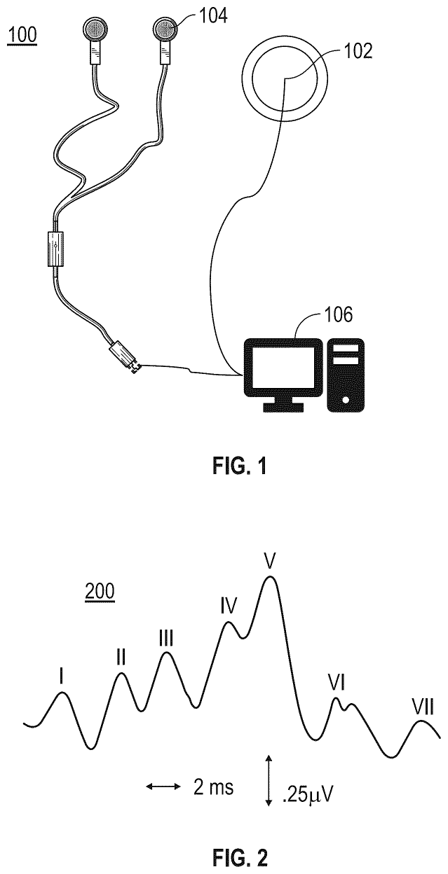

An embodiment of the invention, as shown and described by the various figures and accompanying text, provides systems and methods for the diagnosis of ASD, particularly in newborns and infants, but still applicable in all age groups. A schematic representation of a screening tool 100 capable of performing the inventive method is shown in FIG. 1. This screening tool comprises, and in some embodiments consists of, a single electroencephalography (EEG) electrode 102 placed on the patient's forehead and a speaker device 104. In some embodiments, a plurality of EEG electrodes 102 may be utilized. Moreover, the EEG electrode 102 may be secured to the patient's forehead by any means or method as is known in the art, including, but not limited to, adhesives, glues, tapes, static cling, and the like. The speaker device 104 may be any type of device operable to generate an audible sound to be heard by the patient, including, but not limited to, earphones, headphones, bone oscillating/conduction devices, and loudspeakers. Each of the EEG electrode 102 and the speaker device 104 may be coupled to a computerized device 106. The computerized device 106 may be operable to generate audio signals for playback on the speaker device 104. Further, the computerized device may be configured to receive, record, and analyze signals generated by the EEG electrode 102. In some embodiments, stimulus and acquisition parameter scans may be optimized by each experiment and clinical protocol use.

The system 100 further utilizes advanced digital signal processing designed and optimized specifically for newborns. Specifically, the computerized device 106 may be configured to analyze signals generated by the EEG electrode 102 using such advanced DSP and optimization. The result of such processing may produce an ABR response graph 200 as depicted in FIG. 2. The ABR response graph 200 may comprise, and in some embodiments may consist of, five to seven vertex-positive peaks with 0.8 milliseconds (ms) between peaks. Waves I, II, III, IV, V, VI and VII reflect the response in distal spiral ganglia and auditory nerve, proximal auditory nerve, ventral cochlear nucleus in pons, inferior lateral lemniscus in pons, lateral lemniscus in lower mid-brain, medial geniculate nucleus, and primary auditory cortex, respectively. Particularly, waves I, III and V are commonly recorded to evaluate ABRs. Wave V is the largest, which determines hearing thresholds and characterizes the rest of waves. Collectively, the main signal characteristics associated with audiology deficits are: (1) absence of all waves except waves I, II, or III; (2) abnormal extension of I-III, III-V and I-V interpeak intervals; and (3) abnormal increased difference between left and right ear ABR measurements.

Universal newborn hearing screening (UNHS) has the benefits of superior assessment of the auditory function as well as the effective detection of infants with auditory neuropathy. With the benefit of an advanced digital signal processing algorithm to automatically interpret objective pass/refer results, the clinical performance can reach over 96% sensitivity and specificity. The screening protocol contains placing a speaker device on/in the newborn's ear and sending click sounds at 35 dB above normal hearing level (nHL) at a rate of 77 clicks/sec in the right ear and 79 clicks/sec in the left ear. A surface electrode, such as the EEG electrode 102 of FIG. 1, is used to record electrical responses generated by clicks and identify ABR peaks. Replication of 77/79 clicks improves the accuracy of assessment during brainstem responses to stimuli.

UNHS is applied to assess how the cochlea and the brain pathways process, which might be utilized to determine ASD biomarkers by recording the patterns of ABR waveforms (I-VII) under a click stimulus. Slower brain response in infants is also seen in children with ASD, indicating it may be a biomarker of ASD in infancy. Specifically, wave V latency is associated with impaired myelination common in ASD and also is found in prolonged auditory cortical responses in ASD. Aside from wave V latency, V-negative latency and phase of ABR response are potential biomarkers to detect the difference between normal and ASD individuals. V-negative latency is a latency of a negative wave following wave V positive, which is calculated by dividing the standard deviation of the waveform from grand averages. One of the limitations is that the UNHS testing requires low intensities (1 intensity level in 35 dB), which makes it harder to differentiate most wave components.

ABR testing detects the neural pathway of the patient and exams the auditory function integrity, which can be used to evaluate auditory thresholds of infant nervous system disorders. A chirp stimulus serves as an amplitude-modulated tone that synchronizes auditory sensory cortex at certain modulation frequency. The alternations in neural activity can be detected by the chirp stimulus, as abnormalities in neural oscillations are associated with the auditory cortex in ASD. Moreover, lower-, mid-, and higher-frequency areas of the cochlea are stimulated by the chirp simultaneously, suggesting the chirp-evoked ABR has better synchronous response than the click-evoked ABR. A click-evoked ABR is understood as a basic indicator in brainstem transmission time, whereas a chirp-evoked ABR is a preferred approach in threshold estimation. Compared to a corresponding click, a chirp is useful to detect early evoked responses with higher neural synchronization, larger wave V amplitudes and shorter time test in infants. With larger amplitude ABRs, it detects thresholds at low intensity level efficiently and more effectively in newborn screening.

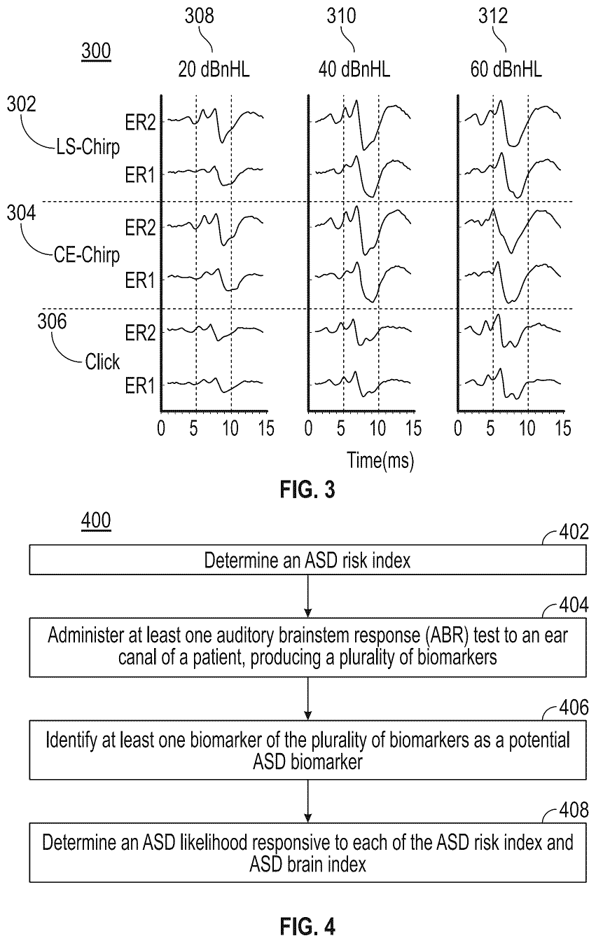

To enhance the ability to identify waveforms under low test administration intensities, a chirp stimulus may be applied to increase waveform amplitudes at the same stimlus intensity. A graph 300 of ABR measurements utilizing chirp stimuli (302, 304) and click stimuli (306) administered at intensities of 20, 40, and 60 decibels above normal hearing level (dB nHL) (308, 310, 312) are presented in FIG. 3. Specifically, 304 represents a chirp 10) stimuli known as “CE-chirps,” popularized by Eberling et al., 302 represents level-specific CE-chirp stimuli, and 306 represents traditional click stimuli as are known in the art. First and second electrical responses (ER1, ER2) are presented for each stimuli 302, 304, 306 and each intensity 308, 310, 312. Chirp stimuli activate the inner ear, notably by improving response detection and reducing threshold of response detection. Consistent with this, a chirp-evoked ABR is less likely to be interfered by background artifact, as it produces bigger neural responses (up to two times) than a brief stimulus (e.g. click ABR). A click ABR that is useful diagnostically has a disadvantage of limited frequency specificity, whereas chirp stimuli provide a broad frequency band range to cover more familiar sounds. In short summary, compared to typical click ABR (UNHS) test 306, the addition of chirp stimuli 302, 304 enables larger amplitude for confident identification of wave I and V, higher signal noise ratio (SNR), extensive frequency band (500-4 k Hz) coverage, the same intensity levels, and accurate ABR threshold. The estimated ABR data collection time from UNHS protocol with chirp stimuli is within a range from 4 to 8 minutes. This demonstrates the UNHS protocol with chirp stimuli it is a safe, cost effective, and efficient screening tool.

Referring now to FIG. 4, a method 400 according to an embodiment of the invention is presented. The method 400 may comprise determining an ASD risk index (ARI) of the patient 402. The ARI may be determined by identifying one or more identifying risk factors associated with the patient. Such identifying risk factors include, but are not limited to, the number of siblings of the patient with ASD, the gestational age of the patient i.e. the number of weeks of gestation the patient was when delivered, and a paternal age, i.e. the 30 age of the father of the patient. In some embodiments, the identifying risk factors are weighted such that one or more identifying risk factor(s) are more consequential to the ARI than one or more of the other identifying risk factors. In some embodiments, the weighted ratio of ASD siblings to gestational age to paternal age is 3:1:1. ARI may be rated using a scale to indicate the comparative risk of ASD. For example, the scale may be from 1-10 with 1 indicating low risk and 10 indicating high risk. As ASD has higher tendency to run in families, higher family risk factors are associated with ASD twins and siblings, earlier birth (e.g. 26 weeks of gestational age), and older paternal age (e.g. >35 years). 80% of identical twins might show co-occurring ASD, while 10-20% of younger siblings of children with ASD have higher risk, especially among families with two siblings. Children born prematurely are more likely to have ASD, suggesting this risk factor is directly correlated with the degree of prematurity. Paternal age plays a vital role ASD risk, which may be affected by maternal age in a non-cooperative manner. Together, family risk factors are correlated with the ASD symptom severity, suggesting ARI can serve as a quantitative indicator in ASD screening and diagnosis.

The method 400 may further comprise determining an ASD brain index (ABI). The ABI may be calculated by administering at least one ABR test to an ear canal of the patient 404. Such an ABR test will produce a plurality of biomarkers. Such biomarkers may include, but are not limited to, the following: phase latencies in one or more of waves I-V; inter-peak latencies between at least one of waves I-III, I-V, or III-V; amplitude; impedance; and noise. As discussed above, the ABR test may comprise a chirp-evoked ABR test. The frequency band of such a test may be within a band from 500 Hz to 4 kHz. The intensity of such a test may have an intensity of less than or equal to at least one of 60 dBnHL, 40 dBnHL, or 20 dBnHL. In some embodiments, the ABR test may include administering a click-evoked ABR test in addition to the chirp-evoked ABR test. The click-evoked ABR test may be administered before or after the chirp-evoked ABR test. The click-evoked ABR test may have an intensity of less or equal to than at least one of 60 dBnHL, 40 dBnHL, or 20 dBnHL. In one embodiment, the click-evoked ABR is administered at an intensity of 35 dbnHL. The click-evoked ABR may be administered at a rate within a range from 70 clicks per second (click rate) to 80 clicks per second. Moreover, the click rate may be different for each ear. In one embodiment, one of the left and right ear may have a click rate of 77 and the other ear may have a click rate of 79.

To estimate the ASD risk index for an individual, a relative ratio is evaluated by comparing biomarkers in an ASD group (a group of diagnosed ASD individuals) with a normal group (reference/baseline of individuals known not to have an ASD diagnosis). For instance, a significant prolong wave V response presents high risk level in ASD. Also, larger phase reflects delayed response time. The larger difference between ASD and normal groups presents higher ASD risk, which can be quantified by ABI. Statistical analyses are applied to examine the changes attributed to ASD biomarkers and prove the significance in early ASD screening and diagnosis. This results in a number of characteristics of biomarkers that indicate an increased risk of ASD that may be used in informing the ASD risk of a patient. Accordingly, the method 400 continues with comparing each biomarker for the patient is then compared with the characteristics of biomarkers that indicate an increased risk of ASD. Any biomarker of the patient that shows characteristics conforming to characteristics of that biomarker indicating an increased risk of ADS may be flagged and identified as such, defining at least one potential ASD biomarker, as shown at step 406. The ABI may be calculated by combining the extent to which each biomarker of the patient conforms to the ADR-associated biomarker characteristics. Such a combination may be made by giving equal weight to each biomarker, or by weighting one or more of the biomarkers. Similar to ARI, the ABI may be on a 1-10 scale with 1 indicating a low ASD risk and 10 a high ASD risk.

The method 400 may continue at step 408 with determining an ASD likelihood responsive to each of the ARI and the ABI. Cross-correlation analysis may provide a method of association between two indexes. Pearson's correlation coefficient (Pearson's r) may be applied to determine the relationship of indexes. With establishment of ARI and ABI, a combined index (ARI+ABI) is generated to determine the association between ARI and ABI. The positive correlation may be observed in this combined index, indicating a higher ARI is accompanied by a greater ABI.

After selection and extraction of ASD biomarkers, machine learning (ML) models may be applied to classify the differences between normal and ASD samples using multivariate feature selection and detection. The support vector machine (SVM) has been widely used the ML applications, which is a robust and established approach for the ABR classification to initiate training by ML models. Such application of ML models may be used both to facilitate, check, or suggest corrections to the biomarkers identifies at step 406 as well as to improve the accuracy of the biomarkers and characteristics thereof in distinguishing the normal and ASD groups used in step 406

To train ML models, the ABR data may be divided by a ratio of 3:1, with 75% is a training dataset and the remaining 25% of the dataset is later used to test the models as a test dataset. The ABR data may be a collection of data generated by administering ABR tests as described above. To prevent the group imbalance in the data, an ASD group for minority populations may be properly distributed into both training and test sets. Due to the high dimensional feature space, ML models need to find optimal hyperparameters. Considering the theoretical and practical implications, hyperparameter optimization is applied and tested by grid search and Bayesian optimization for SVM. Ten-fold cross-validation is used to reduce the biases and variances to obtain robust ML models performance metrics. As more data are obtained, retrained and fine-turned ML model scans potentially yield better diagnoses, indicating this early diagnostic tool for newborn ASD may have clinical utility in assisting clinicians.

Metrics are used to validate the performance of the ML models. In the early diagnostic tool for newborn ASD, a true positive (TP) correctly identifies the number of cases as ASD (where the clinician marks the data as an ASD condition, and the ML models also classifies it with a high ASD risk). A true negative (TN) correctly classifies a number of cases as normal (where the normal outcomes are found in both ABR waveforms and ML models). A false positive (FP) incorrectly represents the number of cases as ASD using ML models. A false negative (FN) incorrectly recognizes the number of cases as normal using ML models. Accuracy is defined as the percentage of correctly classified cases. Sensitivity is defined as the proportion of TP in ASD groups. Specificity is defined as the percentage of TN in normal groups. Precision is computed by the proportion of TP in the sum of TP and FP. High performance in ASD diagnosis is determined by reducing the FP and FN. Each of these metrics are calculated as follows:

$$Accuracy = ( TP + TN ) / ( TP + TN + FP + FN )$$

$$Sensitivity = ( TP / ( TP + FN )$$

$$Specificity = ( TN ) / ( TN + FP )$$

$$Precision = ( TP ) / ( TP + FP )$$

These metrics may be used to measure the quality of the models generated by the SVM ML models and determine whether they represent an improvement on the ML models currently used for identifying biomarkers and determining the ABI for a patient. Selection of the ML model may be made by combining the above metrics into a score, either of even weight or weighting one or more metrics, and comparing the score of the newly generated model with the score of the model presently being used.

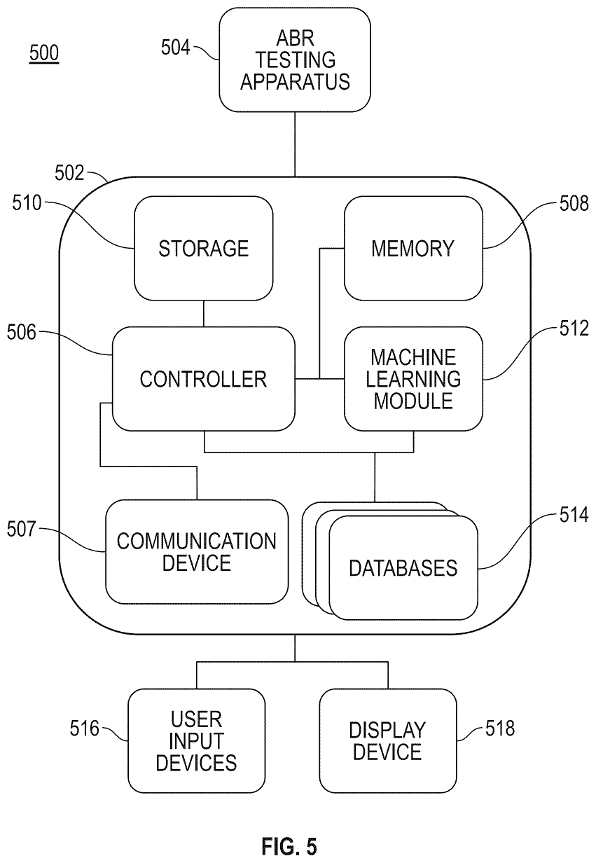

Referring now to FIG. 5, a system 500 according to an embodiment of the invention is presented. The system 500 may comprise computerized device 502 and an ABR testing apparatus 504 substantially as described above and shown in FIG. 1. The ABR testing apparatus 504 may be operably connected to the computerized device 502 such that its operation is controlled by the computerized device 502 and all data generated thereby is provided to the computerized device 502. The connection between the computerized device 502 and the ABR testing apparatus 504 may be by any electronic communication means as is known in the art, including, but not limited to, wired communication standards, such as Universal Serial Bus (USB) or Ethernet, and wireless communication standards, including all IEEE 802.xx standards, such as Wi-Fi, Bluetooth, Zigbee, Z-Wave, and the like. In some embodiments, the computerized device 502 and the ABR testing apparatus may be directly connected, for instance, by USB cable or Wi-Fi Direct. In other embodiments, they may be connected across a network, such as personal area network (PAN), a local area network (LAN), or a wide area network (WAN), such as the Internet. Accordingly, the computerized device 502 may comprise a communication device 507 operable to communicate with at least the ABR testing apparatus 504, such as a USB card, an Ethernet card, or any other network communication card operable to communicate using any of the above referenced communication protocols. In some embodiments, the computerized device 502 may comprise multiple communication devices 507, for instance, a USB card and a network communication card.

The computerized device 502 may comprise all componentry necessary to control the operation of the ABR testing apparatus 504 as well as receive all additional information necessary to perform the ASD diagnosis as described above. Such componentry may include a controller 506, such as a microprocessor, central processing unit, field programmable gate array (FPGA), or any other processing device as is known in the art.

The computerized device 502 may further comprise a memory device 508. The memory device 508 may be a transitory storage medium, such as RAM, SRAM, DRAM, or any other transitory medium as is known in the art. The memory device 508 may be a non-transitory storage medium, such as flash memory, solid state drives, hard disk drives, and the like. Additionally, the computerized device 502 may comprise a discrete non-transitory storage medium 510 that may be any type of non-transitory digital storage device as described. The controller 506 may be operably connected to each of the memory device 508 and the storage 510. At least one of the memory device 508 and the storage 510 may have comprised thereon software configured to cause the controller 506 to at least receive data from the ABR testing apparatus 504, and in some embodiments control the operation of the ABR testing apparatus. The software may further be configured to receive all data necessary to compute the ARI, ABI, and ASD risk index as described above and to compute those values. Such data may be received in any way as is known in the art, including, but not limited to, receiving user input from a user input device 516, such as a keyboard, touchscreen, mouse, or any other input device as is known in the art, or from the communication device 507, for example from a USB flash drive plugged into a USB card comprised by the computerized device 502 or from a remote computerized device that transmits the data across a network via a network communication card comprised by the computerized device. Moreover, the software may further be configured to cause the controller 506 to display the results of computing the ASD risk index and may further display data received from the ABR testing apparatus, the ARI, the ABI, and any other information related to calculating the ASD risk index, on a display device 518.

The computerized device 502 may further comprise a machine learning module 512 and one or more databases 514. Each may be in communication with the controller 506. The machine learning module 512 may be operable to perform the machine learning analysis as described above. The one or more databases 514 may comprise at least the ABR data described above necessary for the machine learning module 512 to generate new models, including the learning data and the test data. The one or more databases 514 may further have stored thereon all data gathered necessary to computer the ARI, ABI, and ASD risk indexes as described above.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

1. A method for early diagnosis of autism spectrum disorder (ASD) comprising:
determining an ASD risk index;
determining an ASD brain index comprising:
administering at least one auditory brainstem response (ABR) test to an ear canal of a patient, producing a plurality of biomarkers; and
identifying at least one biomarker of the plurality of biomarkers as a potential ASD biomarker; and
determining an ASD likelihood responsive to each of the ASD risk index and ASD brain index.

2. The method of claim 1 wherein the plurality of biomarkers comprises wave latencies, inter-peak latencies, and phases.

3. The method of claim 1 wherein determining the ASD risk index comprises identifying risk factors comprising:
determining a number of siblings of the patient with ASD;
determining a gestational age of the patient; and
determining a paternal age associated with the patient.

4. The method of claim 3 wherein the risk factors are weighted by a ratio of 3:1:1 of number of siblings of the patient with ASD to gestational age of the patient to paternal age associated with the patient.

5. The method of claim 1 wherein the ABR test is a chirp-evoked ABR test.

6. The method of claim 4 wherein a frequency band of the ABR test is within a range from 500 Hz to 4 kHz.

7. The method of claim 4 wherein the ABR test has an intensity of less than 60 dBnHL.

8. The method of claim 1 wherein identifying at least one biomarker of the plurality of biomarkers as a potential ASD biomarker comprises:
applying a machine learning model to the plurality of biomarkers; and
receiving an indication of a biomarker of the plurality of biomarkers is a potential ASD biomarker from the application of the machine learning model.

9. The method of claim 8 wherein the machine learning model is trained using ABR data comprising data having biomarkers consistent with baseline biomarker values and data having biomarkers consistent with indicating ASD.

10. The method of claim 9 wherein the ABR data comprises data from members of a minority group with ASD.

11. The method of claim 9 further comprising retraining the machine learning model with ABR data collected after initial training of the machine learning model to produce a retrained machine learning model.

12. The method of 11 wherein the performance of the retrained machine learning model is validated by calculating a metric being at least one of the accuracy, the sensitivity, the specificity, and the precision of the retrained machine learning model and the machine learning model and comparing the metrics of the retrained machine learning model and the machine learning model.

13. The method of claim 12 wherein the performance of the retrained machine learning model is validated by:
calculating the accuracy of each of the retrained machine learning model and the machine learning model by determining the quotient of the sum of the number of true positive results (TP) and true negative results (TN) divided by the sum of the TP, the TN, the false positive results (FP), and the false negative results (FN) of each respective model;
calculating the sensitivity of each of the retrained machine learning model and the machine learning model by determining the quotient of the TP divided by the sum of the TP and the FN;
calculating the specificity of each of the retrained machine learning model and the machine learning model by determining the quotient of the TN by divided by the sum of the TN and the FP;
calculating the precision of each of the retrained machine learning model and the machine learning model by determining the quotient of the TP divided by the sum of the TP and the FP;
determining a score for each respective machine learning model by combining the accuracy, sensitivity, specificity, and precision of each respective machine learning model; and
comparing the score of the retrained machine learning model to the score of the machine learning model.

14. A method for early diagnosis of autism spectrum disorder (ASD) comprising:
determining an ASD risk index by identifying risk factors, the risk factors comprising:
determining a number of siblings of the patient with ASD;
determining a gestational age of the patient; and
determining a paternal age associated with the patient;
determining an ASD brain index comprising:
administering at least one chirp-evoked auditory brainstem response (ABR) test to an ear canal of a patient, producing a plurality of biomarkers; and
identifying at least one biomarker of the plurality of biomarkers as a potential ASD biomarker; and
determining an ASD likelihood responsive to each of the ASD risk index and ASD brain index.

15. The method of claim 14 wherein the plurality of biomarkers comprises wave latencies, inter-peak latencies, and phases.

16. The method of claim 14 wherein the risk factors are weighted by a ratio of 3:1:1 of number of siblings of the patient with ASD to gestational age of the patient to paternal age associated with the patient.

17. The method of claim 14 wherein a frequency band of the ABR test is within a range from 500 Hz to 4 KHz.

18. The method of claim 17 wherein the ABR test has an intensity of less than 60 dBnHL.

19. The method of claim 14 wherein identifying at least one biomarker of the plurality of biomarkers as a potential ASD biomarker comprises:
applying a machine learning model to the plurality of biomarkers; and
receiving an indication of a biomarker of the plurality of biomarkers is a potential ASD biomarker from the application of the machine learning model.

20. The method of claim 19 wherein the machine learning model is trained using ABR data comprising data having biomarkers consistent with baseline biomarker values and data having biomarkers consistent with indicating ASD.

21. The method of claim 20 wherein the ABR data comprises data from members of a minority group with ASD.

22. The method of claim 20 further comprising retraining the machine learning model with ABR data collected after initial training of the machine learning model to produce a retrained machine learning model.

23. The method of 22 wherein the performance of the retrained machine learning model is validated by calculating a metric being at least one of the accuracy, the sensitivity, the specificity, and the precision of the retrained machine learning model and the machine learning model and comparing the metrics of the retrained machine learning model and the machine learning model.

24. The method of claim 23 wherein the performance of the retrained machine learning model is validated by:
calculating the accuracy of each of the retrained machine learning model and the machine learning model by determining the quotient of the sum of the number of true positive results (TP) and true negative results (TN) divided by the sum of the TP, the TN, the false positive results (FP), and the false negative results (FN) of each respective model;
calculating the sensitivity of each of the retrained machine learning model and the machine learning model by determining the quotient of the TP divided by the sum of the TP and the FN;
calculating the specificity of each of the retrained machine learning model and the machine learning model by determining the quotient of the TN by divided by the sum of the TN and the FP;
calculating the precision of each of the retrained machine learning model and the machine learning model by determining the quotient of the TP divided by the sum of the TP and the FP;
determining a score for each respective machine learning model by combining the accuracy, sensitivity, specificity, and precision of each respective machine learning model; and
comparing the score of the retrained machine learning model to the score of the machine learning model.
